SEQUENCE LISTING (16 sequences)

SEQ ID NO: 1 - DNA, 2211 bp, Dependovirus sp.
  source 1..2211: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 2 - protein, 736 aa, Dependovirus sp.
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - DNA, 1234 bp, synthetic construct
  source 1..1234: mol_type = other DNA
gaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 4 - protein, 735 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - DNA, 2211 bp, synthetic construct
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtcaatcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 6 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSQSTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - DNA, 2208 bp, synthetic construct
  source 1..2208: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcctcgaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 8 - protein, 735 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 9 - DNA, 39 bp, synthetic construct
  source 1..39: mol_type = other DNA
aagcaaatctccagccaatcaggagcctcgaacgacaac

SEQ ID NO: 10 - protein, 13 aa, synthetic construct
KQISSQSGASNDN

SEQ ID NO: 11 - DNA, 42 bp, synthetic construct
  source 1..42: mol_type = other DNA
cgagagatcaaaagcggctccgtcgacggaagcaacgccaac

SEQ ID NO: 12 - protein, 14 aa, synthetic construct
REIKSGSVDGSNAN

SEQ ID NO: 13 - DNA, 42 bp, synthetic construct
  source 1..42: mol_type = other DNA
aagcaaatctccagtgcttcaacgggggccagcaacgacaac

SEQ ID NO: 14 - protein, 14 aa, synthetic construct
KQISSASTGASNDN

SEQ ID NO: 15 - DNA, 45 bp, synthetic construct
  source 1..45: mol_type = other DNA
aagcaaatctccaacgggacatcgggaggagccaccaacgacaac

SEQ ID NO: 16 - protein, 15 aa, synthetic construct
KQISNGTSGGATNDN

(19) United States
(12) Patent Application Publication
(10) Pub. No.: US 2026/0234665 A1
(43) Pub. Date: Aug. 13, 2026

(54) MODIFIED CAPSID PROTEINS FOR ENHANCED INTRA-ARTICULAR DELIVERY OF ADENOVIRUS-ASSOCIATED VIRUS (AAV) VECTORS

(71) Applicant: The University of North Carolina at Chapel Hill, Chapel Hill, NC (US)

(72) Inventors: Chengwen LI, Hillsborough, NC (US); Wenjun LI, Chapel Hill, NC (US); Richard Jude SAMULSKI, Hillsborough, NC (US)

(21) Appl. No.: 19/475,126

(22) PCT Filed: Apr. 17, 2024
(86) PCT No.: PCT/US2024/024843

§ 371 Date: Oct. 14, 2025

Related U.S. Application Data
(60) Provisional application No. 63/496,532, filed on Apr. 17, 2023.

(51) Int. Cl. C12N 15/86; A61K 48/00; A61P 19/02

(52) U.S. Cl. CPC C12N 15/86; A61K 48/005; A61P 19/02; C12N 2750/14122; C12N 2750/14133

(57) ABSTRACT

This invention relates to modified adenovirus-associated virus (AAV) capsid proteins with enhanced transduction efficiency and enhanced ability to evade neutralizing antibodies, AAV vectors comprising the same, and methods of using the same for delivery of nucleic acids to a cell of a joint or a joint of a subject.

### STATEMENT OF PRIORITY

This application claims the benefit of U.S. Provisional Application Ser. No. 63/496,532, filed Apr. 17, 2023, the entire contents of which are incorporated by reference herein.

### STATEMENT REGARDING ELECTRONIC FILING OF A SEQUENCE LISTING

A Sequence Listing in XML format, entitled 5470-950WO_ST26.xml, 25,785 bytes in size, generated on Apr. 12, 2024, and filed herewith, is hereby incorporated by reference in its entirety for its disclosures.

### FIELD OF THE INVENTION

This invention relates to modified adenovirus-associated virus (AAV) capsid proteins with enhanced transduction efficiency in joints and enhanced ability to evade neutralizing antibodies, AAV vectors comprising the same, and methods of using the same for delivery of nucleic acids to a cell of a joint or to a joint of a subject.

### BACKGROUND OF THE INVENTION

Arthritis is both a common and incurable group of inflammatory musculoskeletal conditions involving different joints in the body. Rheumatoid arthritis (RA) and osteoarthritis (OA) are the most common types of arthritis. Arthritis can cause significant pain and discomfort, which impacts daily activities. Current therapies for arthritis have aimed at reducing synovial inflammation and pain and preventing joint destruction by targeting pro-inflammatory cytokines or immune cells. The introduction of tumor necrosis factor (TNF) antagonists and other biologics has dramatically improved arthritis treatment, especially in patients with RA. However, a majority of patients do not respond to these drugs. Additionally, these biologics are very expensive and systemic administration induces some severe complications, such as bacterial infection.

Intra-articular (IA) delivery of biologics can lower the likelihood of adverse events in non-target organs and maximize the concentration of the therapeutics in the joints at a lower cost. However, the effectiveness of IA therapy with biologics is greatly impacted due to the rapid clearance of biologics and their efficiency in the synovial space. It is difficult to achieve a sustained and therapeutic concentration of a drug in the affected joints. Moreover, a long-term therapeutic effect requires repeated administration of drugs regardless of delivery routes.

Preventing progression or delaying the onset of arthritis and other joint issues with sustained effective treatment in affected joints without deleterious systemic effects is needed in the art.

### SUMMARY OF THE INVENTION

The present invention provides modified adenovirus-associated virus (AAV) capsid proteins that have improved characteristics and are suitable for generating vectors with a wide variety of uses, including treatment and/or delaying onset of arthritis and other joint issues. A set of novel AAV capsids with modification (e.g., deletion and/or substitution) to variable region 1 (VR1) were rationally engineered to provide a more effective AAV vector for joint-targeted gene delivery. The modified AAV capsids were found to exhibit enhanced joint transduction efficiency and enhanced ability to evade neutralizing antibodies, without crossing the local barrier and entering the blood, after intra-articular injection. These results demonstrate that VR1, specifically VR1 from AAV6, plays an important role in joint transduction efficiency and ability to evade neutralizing antibodies.

One aspect of the invention relates to AAV capsid protein comprising a capsid protein amino acid sequence from AAV6, wherein the VR1 loop comprising amino acid residues 258 to 271 of AAV6 capsid protein is modified by deletion and/or substitution of one or more amino acid residues, wherein the capsid protein comprising the modification provides to an AAV vector comprising the capsid protein increased transduction efficiency and increased ability to evade neutralizing antibodies when administered intra-articularly, relative to an AAV vector comprising an unmodified (i.e., wild-type) AAV6 capsid protein.

An additional aspect of the invention relates to a polynucleotide encoding the capsid protein of the invention, an AAV capsid comprising the capsid protein of the invention, an AAV vector comprising the capsid protein of the invention, and a pharmaceutical composition comprising the AAV vector of the invention.

A further aspect of the invention relates to a method of delivering a nucleic acid to a cell of a joint, the method comprising contacting the cell with the AAV vector of the invention or the pharmaceutical composition of the invention under conditions sufficient for the nucleic acid to enter the cell.

Another aspect of the invention relates to a method of delivering a nucleic acid to a joint of a subject, the method comprising administering intra-articularly to the subject the AAV vector of the invention or the pharmaceutical composition of the invention.

An additional aspect of the invention relates to a method for treating and/or delaying onset of arthritis in a subject in need thereof, comprising administering to the subject an effective amount of the AAV vector of the invention or the pharmaceutical composition of the invention, thereby treating and/or delaying onset of arthritis in the subject.

A further aspect of the invention relates to a method of producing a recombinant AAV particle, comprising providing to a cell permissive for AAV replication: (a) a recombinant AAV template comprising (i) a heterologous nucleic acid, and (ii) at least one inverted terminal repeat; and (b) a polynucleotide comprising replication protein coding sequence(s) and sequence(s) encoding the capsid protein of the invention; under conditions sufficient for the replication and packaging of the recombinant AAV template; whereby recombinant AAV particles are produced in the cell.

These and other aspects of the invention are set forth in more detail in the description of the invention below.

### BRIEF DESCRIPTION OF THE DRAWINGS

FIGS. 1A-1B show transduction efficiency with different AAV serotypes in the joints of mice. 1×10⁹ vg of AAV/luc vectors from different serotypes were administered into the knee joints of C57BL mice. At indicated time points, imaging was taken. In FIG. 1A, the representative imaging was taken at week 6 post AAV injection. FIG. 1B shows the transgene expression in joints with AAV serotypes at different time points. Data is shown as mean±SEM (n=5).

FIG. 2 shows in vitro analysis of luciferase activity in cell lysate of AAV transduced joints. Mice used in FIGS. 1A-1B were euthanized at week 6 after intra-articular injection and the joints harvested for luciferase activity analyses. Data is shown as mean±SEM (n=5).

FIGS. 3A-3B show AAV genome copy number in mouse knees. Knee joint tissues were collected for DNA extraction at 6 weeks post-AAV injection. AAV copy number was detected by qPCR using primers for luciferase. In FIG. 3A, data is shown as mean±SEM (n=4). The relative luciferase expression was calculated by luciferase activity in cell lysate divided by the AAV genome copy number. In FIG. 3B, data is shown as mean±SEM (n=3).

FIG. 4 shows AAV genome copy number in the liver. At week 6 post intra-articular injection of AAV vectors, the liver was harvested for detection of AAV genome.

FIGS. 5A-5B show cell tropisms in joints after intra-articular injection of AAV vectors. Knee joints were sectioned and stained with rabbit anti luciferase antibody. FIG. 5A shows representative images from immunohistochemistry (IHC) staining of mouse knee joints treated with AAV2, AAV5, AAV6 and AAV8. Positive cells were stained as brown color. FIG. 5B shows the efficiency of AAV transduced synoviocytes and chondrocytes. Data is shown as mean±SEM (n=3).

FIG. 6 shows a western blot for capsids from constructs with VR1 substitution. Plasmids with AAV capsid VR1 substitution were transfected into 293 cells. 24 hours later, the cells were collected and cell lysate was used for western blot using antibody B1 to recognize the c-terminal of capsid proteins.

FIG. 7 shows virus yield from VR1 mutants. After transfection of VR1 mutants, AAV titer in supernatant and cell lysates was quantified using qPCR.

FIGS. 8A-8B show transduction efficiency of VR1 mutants in the joints. AAV/Luc vectors from different mutants with VR1 substitution at a dose of 1×10⁹ vg were administered into the knee joints of C57BL mice. At weeks 1 and 6 post AAV injection, imaging was taken. FIG. 8A shows representative imaging at week 6 post AAV injection. FIG. 8B shows transgene expression in joints with AAV VR1 mutants at weeks 1 and 6. Data is shown as mean±SEM (n=4).

FIG. 9 shows an in vitro luciferase assay for joints treated with AAV VR1 mutants. At week 6 post-AAV injection, knee joints were collected and homogenized in passive lysis buffer. The cell lysate was then used to measure luciferase activity. Data is shown as mean±SEM (n=4).

FIGS. 10A-10C show AAV genome copy number in mouse knee joints treated with AAV VR1 mutants. FIG. 10A shows AAV genome copy number (n=3), FIG. 10B shows the relative luciferase expression per AAV genome copy number, and FIG. 10C shows AAV genome copy number in the liver. Data is shown as mean±SEM (n=3).

FIGS. 11A-11B show cell tropism in joints after intra-articular injection of AAV VR1 mutants. FIG. 11A shows representative images from IHC staining of mouse knee joints treated with AAV mutants with VR1 substitution, and FIG. 11B shows the efficiency of AAV transduced synoviocytes and chondrocytes. Data is shown as mean±SEM (n=3).

FIGS. 12A-12B show transduction efficiency of AAV6D and AAV6M/luc in joints in vivo. FIG. 12A shows representative imaging, while FIG. 12B shows transduction efficiency in joints treated with AAV6D and 6M at week 6.

FIG. 13 shows an in vitro luciferase assay for joints treated with AAV6D and AAV6M, wherein the luciferase activity in joint lysate was analyzed in vitro at week 6 post-AAV injection.

FIGS. 14A-14C show AAV genome copy number in mouse knee joints treated with AAV6D and AAV6M mutants. FIG. 14A shows AAV genome copy number (n=3), FIG. 14B shows AAV genome copy number in the liver, and FIG. 14C shows the relative luciferase expression per AAV genome copy number in the joints. Data is shown as mean±SEM (n=3).

FIGS. 15A-15B show cell tropism in joints after intra-articular injection of AAV6D or AAV6M mutants. FIG. 15A shows representative images from IHC staining of mice knee joints treated with AAV6D or AAV6M. FIG. 15B shows the efficiency of AAV transduced synoviocytes and chondrocytes. Data is shown as mean±SEM (n=3).

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations, and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., PatentIn User Manual, 99-102 (November 1990) (U.S. Patent and Trademark Office).

Except as otherwise indicated, standard methods known to those skilled in the art may be used for the construction of recombinant AAV (rAAV) constructs, packaging vectors expressing the parvovirus Rep and/or Cap sequences, and transiently and stably transfected packaging cells. Such techniques are known to those skilled in the art. See, e.g., GREEN et al., MOLECULAR CLONING: A LABORATORY MANUAL 4th Ed. (Cold Spring Harbor, NY, 2012); AUSUBEL et al., CURRENT PROTOCOLS IN MOLECULAR BIOLOGY (Green Publishing Associates, Inc. and John Wiley & Sons, Inc., New York).

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention (e.g., rAAV replication). Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in transduction activity of at least about 50% or more as compared to the activity of a polypeptide consisting of the recited sequence.

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 1.

TABLE 1

| | |
|---|---|
| AAV Serotypes/Isolates | GenBank Accession Number |

Complete Genomes

| | |
|---|---|
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, NC_004828 |
| Avian AAV strain DA-1 | NC_006263, AY629583 |
| Bovine AAV | NC_005889, AY388617 |

Clade A

| | |
|---|---|
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |

Clade B

| | |
|---|---|
| Hu19 | AY530584 |
| Hu20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |

Clade B

| | |
|---|---|
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |

Clade C

| | |
|---|---|
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |

Clade D

| | |
|---|---|
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |

Clade E

| | |
|---|---|
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |

Clade F

| | |
|---|---|
| Hu14 (AAV9) | AY530579 |
| Hu31 | AY530596 |
| Hu32 | AY530597 |

Clonal Isolate

| | |
|---|---|
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., FIELDS et al., VIROLOGY, volume 2, chapter 69 (4th ed., Lippincott-Raven Publishers). A number of relatively new AAV serotypes and clades have been identified (See, e.g., Gao et al., (2004) J. Virol. 78:6381; Moris et al., (2004) Virol. 33-:375; and Table 1).

The genomic sequences of various serotypes of AAV as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73:939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-: 375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

The term “tropism” as used herein refers to entry of the virus into the cell, optionally and preferably followed by expression (e.g., transcription and, optionally, translation) of sequences carried by the viral genome in the cell, e.g., for a recombinant virus, expression of the heterologous nucleotide sequences(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of AAV, gene expression from the viral genome may be from a stably integrated provirus, from a non-integrated episome, as well as any other form in which the virus may take within the cell.

As used herein, “transduction” of a cell by AAV refers to AAV-mediated transfer of genetic material into the cell. See, e.g., FIELDS et al., VIROLOGY, volume 2, chapter 69 (3d ed., Lippincott-Raven Publishers).

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotide), and can be either single or double stranded DNA sequences.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” polynucleotide (e.g., an “isolated DNA” or an “isolated RNA”) means a polynucleotide separated or substantially free from at least some of the other components of the naturally occurring organism or virus, for example, the cell or viral structural components or other polypeptides or nucleic acids commonly found associated with the polynucleotide.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

A “therapeutic polypeptide” is a polypeptide that may alleviate or reduce symptoms that result from an absence or defect in a protein in a cell or subject. Alternatively, a “therapeutic polypeptide” is one that otherwise confers a benefit to a subject, e.g., anti-cancer effects or improvement in transplant survivability.

As used herein, the term “modified,” as applied to a polynucleotide or polypeptide sequence, refers to a sequence that differs from a wild-type sequence due to one or more deletions, additions, substitutions, or any combination thereof.

As used herein, by “isolate” or “purify” (or grammatical equivalents) an AAV vector, it is meant that the AAV vector is at least partially separated from at least some of the other components in the starting material.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid” are used interchangeably herein and refer to a sequence that is not naturally occurring in the virus. In some embodiments, the heterologous nucleic acid comprises an open reading frame that encodes a polypeptide or nontranslated RNA of interest (e.g., for delivery to a cell or subject).

As used herein, the terms “AAV vector,” “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone or a plasmid.

A “rAAV vector genome” or “rAAV genome” is an AAV genome (i.e., vDNA) that comprises one or more heterologous nucleic acid sequences. rAAV vectors generally require only the 145 base ITR in cis to generate virus. All other viral sequences are dispensable and may be supplied in trans (Muzyczka (1992) Curr. Topics Microbiol. Immunol. 158:97). Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as the “double-D sequence” as described in U.S. Pat. No. 5,478,745 to Samulski et al. FIG. 24 provides examples of synthetic ITRs contemplated by the present invention.

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like.

The AAV vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

Further, the viral capsid or genomic elements can contain other modifications, including insertions, deletions and/or substitutions.

As used herein, the term “amino acid” encompasses any naturally occurring amino acids, modified forms thereof, and synthetic amino acids. Naturally occurring, levorotatory (L-) amino acids are shown in Table 2.

TABLE 2

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) or can be an amino acid that is modified by post-translation modification (e.g., acetylation, amidation, formylation, hydroxylation, methylation, phosphorylation or sulfatation).

TABLE 3
Amino Acid Residue Derivatives

| Modified Amino Acid Residue | Abbreviation |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Idc |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid as described by Wang et al., (2006) Annu. Rev. Biophys. Biomol. Struct. 35:225-49. These unnatural amino acids can advantageously be used to chemically link molecules of interest to the AAV capsid protein.

The term “template” or “substrate” is used herein to refer to a polynucleotide sequence that may be replicated to produce the adenovirus-associated virus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, AAV “Rep coding sequences” indicate the nucleic acid sequences that encode the AAV non-structural proteins that mediate viral replication and the production of new virus particles. The AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wild-type or synthetic. A wild-type large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the AAV “cap coding sequences” encode the structural proteins that form a functional AAV capsid (i.e., can package DNA and infect target cells). Typically, the cap coding sequences will encode all of the AAV capsid subunits, but less than all of the capsid subunits may be encoded as long as a functional capsid is produced. Typically, but not necessarily, the cap coding sequences will be present on a single nucleic acid molecule.

The capsid structure of autonomous AAV are described in more detail in BERNARD N. FIELDS et al., VIROLOGY, volume 2, chapters 69 & 70 (4th ed., Lippincott-Raven Publishers).

### Modified Adeno-Associated Virus (AAV) Capsid Proteins

The present invention provides modified AAV capsid proteins that provide enhanced tissue transduction capabilities and/or enhanced ability to evade neutralizing antibodies and can be used to prepare AAV vectors for efficient delivery of nucleic acids to cells in a joint, e.g., by IA injection. The inventors have discovered that modifications, such as deletions or substitutions, to the VR1 loop of the capsid protein result in enhanced cell transduction and/or enhanced ability to evade neutralizing antibodies.

One aspect of the invention relates to an AAV capsid protein comprising a capsid protein amino acid sequence from AAV6 (i.e., the capsid protein has a backbone of AAV6 capsid protein), wherein the VR1 loop comprising, consisting essentially of, or consisting of amino acid residues 258 to 271 of AAV6 capsid protein is modified by deletion and/or substitution of one or more amino acid residues, wherein the capsid protein comprising the modification provides to an AAV vector comprising the capsid protein increased transduction efficiency and increased ability to evade neutralizing antibodies when administered IA, relative to an AAV vector comprising an unmodified (wild-type) AAV6 capsid protein. The wild-type AAV6 capsid protein has the amino acid sequence of SEQ ID NO:2.

AAV serotypes are well known in the art and are described above in Table 1.

Capsid protein sequences are known in the art and are available in sequence databases such as GenBank. The amino acid residue numbers used herein with respect to the capsid protein from AAV1, AAV2, AAV3b, AAV4, AAV5, AAV6, AAV7, AAV8, and AAV9 refer to the sequences as disclosed in GenBank Accession Nos. as listed in Table 1.

The VR1 region is an art-recognized portion of the capsid protein sequence of AAV serotypes. The loop that makes up the VR1 region comprises, consists essentially of, or consists of amino acid residues 258 to 271 of AAV6 or the corresponding amino acid residues from another AAV. Because the VR1 loop is a well-recognized structure, the corresponding amino acid residues may be readily identified by one of skill in the art. For example, Table 4 includes a list of exemplary VR1 amino acid residues. While specific residues are listed, it will be understood by one of skill in the art that the boundaries of the VR1 region are approximate and may vary by one or two residues.

TABLE 4

| AAV | |
|---|---|
| Scrctypc | VR1 Aminc Acid Rcsiducs |
| AAV2 | ₂₅₈KQISSQSGASNDN₂₇₀ |
| | (SEQ ID NO: 10) |
| AAV5 | ₂₄₈REIKSGSVDGSNAN₂₆₁ |
| | (SEQ ID NO: 12) |
| AAV6 | ₂₅₈KQISSASTGASNDN₂₇₁ |
| | (SEQ ID NO: 14) |
| AAV8 | ₂₅₉KQISNGTSGGATNDN₂₇₃ |
| | (SEQ ID NO: 16) |

In some embodiments, the transduction efficiency of an AAV vector comprising the modified capsid protein is increased at least about 10% relative to an AAV vector comprising an AAV6 capsid protein that does not contain the modification, e.g., at least about 10%, 20%, 30%, 40%, 50%, 60%, 70%, 80%, 90%, 100%, 150%, 200%, 500%, or more. Transduction efficiency may be measured by techniques well known in the art and as described herein. In certain embodiments, the transduction of joints, e.g., shoulder, knee, and/or knuckle joints, is increased at least about 10% relative to an AAV vector comprising an AAV6 capsid protein that does not contain the modification, e.g., at least about 10%, 20%, 30%, 40%, 50%, 60%, 70%, 80%, 90%, 100%, 150%, 200%, 500%, or more.

In some embodiments, the capsid is modified by deleting or substituting one or more amino acid residues of amino acid residues 258 to 271 of AAV6 capsid protein, e.g., 1, 2, 3, 4, 5, 6, 7, 8, 9, or 10 or more residues are deleted or substituted. In certain embodiments, the capsid is modified by deleting one or more amino acid residues of amino acid residues 258 to 271 of AAV6 capsid protein, e.g., 1, 2, 3, 4, 5, 6, 7, 8, 9, or 10 or more residues are deleted. In some embodiments, the capsid is modified by deleting amino acid residue threonine 265 of AAV6 capsid protein. In another embodiment, the capsid is modified by substituting one or more amino acid residues of amino acid residues 258 to 271 of AAV6 capsid protein, e.g., 1, 2, 3, 4, 5, 6, 7, 8, 9, or 10 or more residues are substituted. In further embodiments, the capsid is modified by substituting the VR1 loop of AAV6 capsid protein with the VR1 loop of AAV2 capsid protein (amino acid residues 258-270, as shown in Table 4). In some embodiments, the capsid is modified by substituting amino acid residue alanine 263 of AAV6 capsid protein with glutamine. In another embodiment, the capsid is modified by deleting amino acid residue threonine 265 of AAV6 capsid protein and substituting amino acid residue alanine 263 of AAV6 capsid protein with glutamine.

In certain embodiments, the modification is made to an AAV6 capsid, resulting in an increase in transduction of joints. In certain embodiments, the modification is one or more of the modifications shown in Table 5. In some embodiments, the capsid protein comprises, consists essentially of, or consists of an amino acid sequence encoded by the nucleotide sequence of SEQ ID NO: 3. In some embodiments, the capsid protein comprises, consists essentially of, or consists of an amino acid sequence encoded by a nucleotide sequence that is at least 80% identical, e.g., at least 85%, 90%, 95%, 96%, 97%, 98%, or 99% identical, to SEQ ID NO:3. In some embodiments, the capsid protein comprises, consists essentially of, or consists of an amino acid sequence encoded by the nucleotide sequence of SEQ ID NO:5. In some embodiments, the capsid protein comprises, consists essentially of, or consists of an amino acid sequence encoded by a nucleotide sequence that is at least 80% identical, e.g., at least 85%, 90%, 95%, 96%, 97%, 98%, or 99% identical, to SEQ ID NO:5. In some embodiments, the capsid protein comprises, consists essentially of, or consists of an amino acid sequence encoded by the nucleotide sequence of SEQ ID NO:7. In some embodiments, the capsid protein comprises, consists essentially of, or consists of an amino acid sequence encoded by a nucleotide sequence that is at least 80% identical, e.g., at least 85%, 90%, 95%, 96%, 97%, 98%, or 99% identical, to SEQ ID NO:7. In some embodiments, the capsid protein comprises, consists essentially of, or consists of the amino acid sequence of one of SEQ ID NOS: 4, 6, or 8 or a sequence at least 80% identical, e.g., at least 85%, 90%, 95%, 96%, 97%, 98%, or 99% identical to one of SEQ ID NOS: 4, 6, or 8.

TABLE 5
Variable region 1 mutations.

| Serotype | Mutations |
|---|---|
| AAV6D | T265del |
| SEQ ID NO: 4 | |
| AAV6M | 263A > Q |
| SEQ ID NO: 6 | |
| AAV62 | T265del, 263A > Q |
| SEQ ID NO: 8 | |

In certain embodiments, the modification is made to an AAV6 capsid, resulting in an increase in transduction of joints. In certain embodiments, the transduction of joints is increased at least about 10% relative to an AAV vector comprising a capsid protein that does not contain the modification, e.g., at least about 10%, 20%, 30%, 40%, 50%, 60%, 70%, 80%, 90%, 100%, 150%, 200%, 500%, or more. In certain embodiments, the modification is made to an AAV6 capsid, resulting in an increase in ability to evade neutralizing antibodies when administered intra-articularly. In some embodiments, the modification is made to an AAV6 capsid, resulting in an increase in transduction of joints and an increase in ability to evade neutralizing antibodies when administered intra-articularly.

One aspect of the invention relates to a polynucleotide encoding the capsid protein of the invention. In some embodiments, the polynucleotide comprises, consists essentially of, or consists of a nucleotide sequence encoding the capsid protein of the invention, e.g., one of the sequences disclosed herein. In other embodiments, the polynucleotide comprises, consists essentially of, or consists of a nucleotide sequence encoding a capsid protein that is at least 80% identical, e.g., at least 85%, 90%, 95%, 96%, 97%, 98%, or 99% identical to one of the capsid protein sequences disclosed herein. In some embodiments, the polynucleotide comprises, consists essentially of, or consists of a nucleotide sequence that is at least 80% identical, e.g., at least 85%, 90%, 95%, 96%, 97%, 98%, or 99% identical to one of the polynucleotide sequences disclosed herein.

An additional aspect of the invention relates to an AAV capsid comprising the capsid protein of the invention. In some embodiments, all of the capsid proteins in the capsid are capsid proteins of the invention. In other embodiments, some but not all of the capsid proteins in the capsid are capsid proteins of the invention.

The invention also provides an AAV vector comprising the AAV capsid protein of the invention. The AAV vector may further comprise a nucleic acid comprising a recombinant viral template, wherein the nucleic acid is encapsidated by the AAV capsid. In some embodiments, the AAV vector comprises a recombinant viral template comprising a heterologous nucleic acid of interest. In some embodiments, the AAV vector comprises a recombinant viral template comprising a promoter. The promoter may be tissue specific, such as specific to chondrocytes and/or synoviocytes. The invention further provides a recombinant AAV particle comprising the capsid protein of the invention. AAV vectors and recombinant AAV particles are discussed further below.

In certain embodiments, the AAV vector exhibits a modified leakage from a joint into the blood and/or liver due to the presence of the capsid protein of the invention. In one embodiment, the AAV vector exhibits minimized leakage from a joint into the blood and/or liver, compared to an AAV vector comprising a wild-type capsid protein. The term “minimized leakage,” as used herein, refers an amount of leakage that is less than 50%, 40%, 30%, 20%, or 10% of the leakage of an AAV vector that does not have the capsid protein of the invention.

### Methods of Producing AAV Vectors

The present invention further provides methods of producing AAV vectors. In one particular embodiment, the present invention provides a method of producing a recombinant AAV particle, comprising providing to a cell permissive for AAV replication: (a) a recombinant AAV template comprising (i) a heterologous nucleic acid, and (ii) at least one inverted terminal repeat (ITR); and (b) a polynucleotide comprising replication protein coding sequence(s) and sequence(s) encoding the capsid protein of the invention; under conditions sufficient for the replication and packaging of the recombinant AAV template; whereby recombinant AAV particles are produced in the cell. Conditions sufficient for the replication and packaging of the recombinant AAV template can be, e.g., the presence of AAV sequences sufficient for replication of the AAV template and encapsidation into AAV capsids (e.g., AAV rep sequences and AAV cap sequences) and helper sequences from adenovirus and/or herpesvirus. In particular embodiments, the AAV template comprises two AAV ITR sequences, which are located 5′ and 3′ to the heterologous nucleic acid sequence, although they need not be directly contiguous thereto.

In some embodiments, the recombinant AAV template comprises an ITR that is not resolved by Rep to make duplexed AAV vectors as described in international patent publication WO 01/92551.

The AAV template and AAV rep and cap sequences are provided under conditions such that AAV vector comprising the AAV template packaged within the AAV capsid is produced in the cell. The method can further comprise the step of collecting the AAV vector from the cell. The AAV vector can be collected from the medium and/or by lysing the cells.

The cell can be a cell that is permissive for adenovirus-associated virus viral replication. Any suitable cell known in the art may be employed. In particular embodiments, the cell is a mammalian cell (e.g., a primate or human cell). As another option, the cell can be a trans-complementing packaging cell line that provides functions deleted from a replication-defective helper virus, e.g., 293 cells or other E1a trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell.

Typically the AAV rep/cap sequences will not be flanked by the TRs, to prevent rescue and/or packaging of these sequences.

The AAV template can be provided to the cell using any method known in the art. For example, the template can be supplied by a non-viral (e.g., plasmid) or viral vector. In particular embodiments, the AAV template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the AAV template is provided by a replicating rAAV virus. In still other embodiments, an AAV provirus comprising the AAV template is stably integrated into the chromosome of the cell.

To enhance virus titers, helper virus functions (e.g., adenovirus or herpesvirus) that promote a productive adenovirus-associated virus infection can be provided to the cell. Helper virus sequences necessary for adenovirus-associated virus replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient adenovirus-associated virus production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by ITRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector can further comprise the AAV template. The AAV rep/cap sequences and/or the AAV template can be inserted into a deleted region (e.g., the E1a or E3 regions) of the adenovirus.

In a further embodiment, the AAV rep/cap sequences and the adenovirus helper sequences are supplied by a single adenovirus helper vector. According to this embodiment, the AAV template can be provided as a plasmid template.

In another illustrative embodiment, the AAV rep/cap sequences and adenovirus helper sequences are provided by a single adenovirus helper vector, and the AAV template is integrated into the cell as a provirus. Alternatively, the AAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The AAV template can be provided as a separate replicating viral vector. For example, the AAV template can be provided by an AAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the AAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by ITRs so that these sequences are not packaged into the AAV virions.

Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Ther. 6:986 and WO 00/17377.

As a further alternative, the AAV vectors of the invention can be produced in insect cells using baculovirus vectors to deliver the rep/cap genes and AAV template as described, for example, by Urabe et al., (2002) Human Gene Ther. 13:1935-43.

AAV vector stocks free of contaminating helper virus may be obtained by any method known in the art. For example, adenovirus-associated virus and helper virus may be readily differentiated based on size. Adenovirus-associated virus may also be separated away from helper virus based on affinity for a heparin substrate (Zolotukhin et al., (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of adenovirus-associated virus. Adenovirus mutants defective for late gene expression are known in the art (e.g., ts100K and ts149 adenovirus mutants).

The AAV vectors of the present invention are useful for the delivery of nucleic acids to cells in vitro, ex vivo, and in vivo. In particular, the AAV vectors can be advantageously employed to deliver or transfer nucleic acids to animal, including mammalian, cells.

Any heterologous nucleic acid sequence(s) of interest may be delivered in the AAV vectors of the present invention. Nucleic acids of interest include nucleic acids encoding polypeptides, including therapeutic (e.g., for medical or veterinary uses), immunogenic (e.g., for vaccines), or diagnostic polypeptides.

Heterologous nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Alternatively, in particular embodiments of this invention, the heterologous nucleic acid may encode a functional nucleic acid, i.e., a nucleic acid with bioactivity without having to be translated, e.g., a functional RNA, e.g., an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like.

The AAV vector may also comprise a heterologous nucleic acid that shares homology with and recombines with a locus on a host chromosome. This approach can be utilized, for example, to correct a genetic defect in the host cell.

As a further alternative, the heterologous nucleic acid can encode any polypeptide that is desirably produced in a cell in vitro, ex vivo, or in vivo. For example, the AAV vectors may be introduced into cultured cells and the expressed gene product isolated therefrom.

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include joint-specific or joint-preferred. In some embodiments, the promoter/enhancer element for gene delivery can be tissue-specific or -preferred to chondrocytes and/or synoviocytes. Other inducible promoter/enhancer elements include hormone-inducible and metal-inducible elements.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The AAV vectors according to the present invention provide a means for delivering heterologous nucleic acids into a broad range of cells, including dividing and non-dividing cells. The AAV vectors can be employed to deliver a nucleic acid of interest to a cell in vitro, e.g., to produce a polypeptide in vitro or for ex vivo gene therapy. The AAV vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The AAV vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the AAV vectors of the present invention can be employed to deliver a heterologous nucleic acid encoding a polypeptide or functional RNA to treat and/or prevent any disease state for which it is beneficial to deliver a therapeutic polypeptide or functional RNA. In some embodiments, the AAV vectors of the present invention can be employed to deliver a heterologous nucleic acid encoding a polypeptide or functional RNA to treat and/or delay onset of a joint issue. In some embodiments, the AAV vectors of the present invention can be employed to deliver a heterologous nucleic acid encoding a polypeptide or functional RNA to treat and/or delay onset of arthritis. Illustrative disease states include, but are not limited to: joint pain, joint inflammation, joint injury, osteoarthritis, juvenile arthritis, reactive arthritis, gout, psoriatic arthritis, fibromyalgia, ankylosing spondylitis or rheumatoid arthritis, Felty's syndrome, diffuse idiopathic skeletal hyperostosis (DISH), Behçet's disease, inflammatory arthritis, mixed connective tissue disease, infectious arthritis, Granulomatosis with polyangiitis (GPA), Mixed Connective Tissue disease (MCTD), myositis (dermatomyositis, polymyositis), Paget's disease, Pseudogout, polymyalgia rheumatica with giant cell arteritis, Raynaud's Phenomenon, scleroderma, systemic lupus erythematosus, and Sjögren syndrome.

The AAV vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

AAV vectors according to the instant invention find use in diagnostic and screening methods, whereby a nucleic acid of interest is transiently or stably expressed in a cell culture system, or alternatively, a transgenic animal model.

The AAV vectors of the present invention can also be used for various non-therapeutic purposes, including but not limited to use in protocols to assess gene targeting, clearance, transcription, translation, etc., as would be apparent to one skilled in the art. The AAV vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

Alternatively, the AAV vector may be administered to a cell ex vivo and the altered cell is administered to the subject. The AAV vector comprising the heterologous nucleic acid is introduced into the cell, and the cell is administered to the subject, where the heterologous nucleic acid can be expressed and induce a response in the subject. In particular embodiments, the cell is an antigen-presenting cell (e.g., a dendritic cell).

### Subjects, Pharmaceutical Formulations, and Modes of Administration

AAV vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles, and adults.

In particular embodiments, the present invention provides a pharmaceutical composition comprising an AAV vector and/or capsid of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid.

By “pharmaceutically acceptable” it is meant a material that is not toxic or otherwise undesirable, i.e., the material may be administered to a subject without causing any undesirable biological effects.

One aspect of the present invention is a method of delivering a nucleic acid to a cell of a joint in vitro, comprising contacting the cell with an AAV vector of the invention or a pharmaceutical composition of the invention, under conditions sufficient for the nucleic acid to enter the cell. The AAV vector may be introduced into the cells of the joint at the appropriate multiplicity of infection according to standard transduction methods suitable for the particular target cells. Titers of AAV vector to administer can vary, depending upon the target cell type and number, and the particular AAV vector, and can be determined by those of skill in the art without undue experimentation. In representative embodiments, at least about 10³ infectious units, more preferably at least about 10⁵ infectious units are introduced to the cell. The cell(s) into which the AAV vector is delivered can be of any type, including but not limited to joint cells. Moreover, the cell can be from any species of origin, as indicated above.

The AAV vector can be introduced into cells in vitro for the purpose of administering the modified cell to a subject. In particular embodiments, the cells have been removed from a subject, the AAV vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant AAV vector can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the AAV vector are administered to the subject in a treatment effective or prevention effective amount in combination with a pharmaceutical carrier.

In some embodiments, the AAV vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of delivering a nucleic acid to a joint of a subject, the method comprising administering IA to the subject an AAV vector of the invention or a pharmaceutical composition of the invention. Administration of the AAV vectors and/or capsids according to the present invention to a human subject or an animal in need thereof can be by any means known in the art. Optionally, the AAV vector and/or capsid is delivered in a treatment effective or prevention effective dose in a pharmaceutically acceptable carrier. IA drug delivery vehicles can include those described in Pharmaceutics, 2021 December; 13(12): 2166; doi: 10.3390/pharmaceutics1312166, incorporated herein by reference, with specific reference to Table 1 (identifying different drug delivery systems investigated for intra-articular osteoarthritis therapy).

The AAV vectors and/or capsids of the invention can further be administered to elicit an immunogenic response (e.g., as a vaccine). Typically, immunogenic compositions of the present invention comprise an immunogenically effective amount of AAV vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the AAV vector and/or capsid to be administered to a subject depend upon the mode of administration, the disease or condition to be treated and/or prevented, the individual subject's condition, the particular AAV vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸-10¹⁵ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include direct tissue or organ injection (e.g., IA injection). In some embodiments, an AAV vector of the invention or a pharmaceutical composition of the invention is administered to a joint in a subject. Administration can be to any site in a subject, including, without limitation, a site selected from the group consisting of a shoulder joint, a knee joint, and a knuckle joint. The most suitable route in any given case will depend on the nature and severity of the condition being treated and/or prevented and on the nature of the particular vector that is being used.

In representative embodiments, the invention is used to treat and/or delay onset of a joint issue. In some embodiments, the joint issue is selected from a group consisting of joint disease, joint pain, joint inflammation, and joint injury.

In representative embodiments, the invention is used to treat and/or delay onset of arthritis in a subject in need thereof, comprising administering to a subject an effective amount of the AAV vector of the invention or the pharmaceutical composition of the invention, thereby treating and/or delaying onset of arthritis in the subject. In particular embodiments, the AAV vector and/or capsid is administered in a liquid formulation by direct injection (e.g., IA injection) to a joint of the subject. In particular embodiments, the AAV vector can be administered to a joint intra-articularly as described elsewhere herein. Joint histopathology may be improved, stabilized without further worsening, or a rate of worsening slowed with the methods and pharmaceutical formulation disclosed herein. By way of example, scoring of joints in subjects with osteoarthritis can be scored according to the Osteoarthritis Research Society International (OARSI) system to determine stability, worsening or improvement of joint histopathology, as is well known in the art. See, e.g., Osteoarthritis Cartilage 19:324-331; Osteoarthritis Cartilage 20:476-485.

Injectables can be prepared in conventional forms, either as liquid solutions or suspensions, solid forms suitable for solution or suspension in liquid prior to injection, or as emulsions. One may administer the AAV vector and/or virus capsids of the invention in a local manner, for example, in a depot or sustained-release formulation. Further, the AAV vector and/or virus capsid can be delivered adhered to a surgically implantable matrix (e.g., as described in U.S. Patent Publication No. 2004-0013645).

In particular embodiments, an AAV vector or pharmaceutical composition of the invention may be administered to a subject who has or is at risk of developing arthritis. In some embodiments, the arthritis is selected from a group consisting of osteoarthritis, juvenile arthritis, reactive arthritis, gout, psoriatic arthritis, fibromyalgia, ankylosing spondylitis or rheumatoid arthritis, Felty's syndrome, diffuse idiopathic skeletal hyperostosis (DISH), Behçet's disease, inflammatory arthritis, mixed connective tissue disease, infectious arthritis, Granulomatosis with polyangiitis (GPA), Mixed Connective Tissue disease (MCTD), myositis (dermatomyositis, polymyositis), Paget's disease, Pseudogout, polymyalgia rheumatica with giant cell arteritis, Raynaud's Phenomenon, scleroderma, systemic lupus erythematosus, and Sjögren syndrome.

Having described the present invention, the same will be explained in greater detail in the following examples, which are included herein for illustration purposes only, and which are not intended to be limiting to the invention.

### Example 1

Arthritis is a group of inflammatory musculoskeletal conditions involving different joints in the body. They usually come with agonizing pain or discomfort that impact daily activities. The current systemic treatments using biological drugs come with various side effects while local treatments often require multiple visits. Due to these drawbacks, IA gene therapy, particularly with AAV vectors, has been explored to address the most severe problems in local sites. There are three main considerations when AAV vectors are used for IA gene delivery: transduction efficiency in the joint; off target transduction in other tissues, and the existence of AAV neutralizing antibodies (Nabs) in joint fluid.

We first screened AAV1-9 vectors encoding firefly luciferase (AAV/luc), 5×10⁹ vg AAV vectors were administered into the knees of C57BL/6 mice. The results from mouse imaging and in vitro luciferase assays showed that AAV6 had the best knee joint transduction, followed by AAV1, 8, 9, 7, 2, and 5, while AAV3 and AAV4 obtained the lowest transduction. The mice administered with AAV7, 8, and 9 vectors also displayed luciferase expression in the liver. We also compared AAV transduction under inflammatory and normal conditions using collagen induced arthritis mouse model (CIA), which showed CIA mice exhibited a higher transduction starting from 3 mo after the priming dose of arthritis induction, until 12 mo (our ending point).

It was reported that VR1 is one of the key determinants for AAV transduction and neutralizing antibody binding. To develop a more effective AAV vector for joint targeted gene delivery, we rationally engineered a set of novel AAV capsids with substitution of VR1 between AAV2, AAV6, and AAV8. The results showed the substitution of VR1 from AAV6 into either AAV2 or AAV8 increased joint transduction when compared to AAV2 or AAV8 before modification. The mutant AAV2-VR1-6 (created by replacing VR1 of AAV2 with that of AAV6) showed a 3-fold higher transduction efficiency than AAV2, and the mutant AAV8-VR1-6 (created by replacing VR1 of AAV8 with that of AAV6) had a 2-fold higher transduction efficiency than AAV8. Interestingly, the mutant AAV6-VR1-8 with substitution of VR1 from AAV8 into the AAV6 capsid had an 8-fold lower transduction efficiency than AAV6, while the mutant AAV6-VR1-2 with swapping of VR1 from AAV2 into AAV6 induced similar joint transduction to AAV6 with no significant difference. It is worth noting that mice injected with AAV6-VR1-2 displayed lower Nab titers against AAV6 and higher transduction efficiency per copy number than AAV6.

There is a difference of only two amino acids between VR1s from AAV2 and AAV6. To further investigate the role of each amino acid in enhancing AAV6 joint transduction, we made two other novel AAV capsids: the mutant AAV6D, with a deletion of the 265 amino acid threonine, and the mutant AAV6M, with a mutation of the 263 amino acid from alanine to glutamine. After direct joint injection, AAV6M experienced a 100-fold decrease in transduction compared to AAV6, while AAV6D induced transduction at an efficiency 2-fold higher than that of AAV6 and AAV6-VR1-2. Additionally, Nab titers in mice injected with AAV6D were low, with a titer of less than 1:10 in mouse serum. Similar to AAV6, the transgene expression in mice receiving AAV6D vectors was mainly restricted to the injected joints, with less than 0.01 viral particles per cell detected in the liver. Immunohistochemistry staining with rabbit anti-luciferase antibody also demonstrated that AAV6D successfully transduced synoviocytes (29.3±4.5%) and chondrocytes (26.8±4.1%). These results from our study demonstrate that VR1 from AAV6 plays an important role in joint transduction efficiency, and that novel AAV mutant AAV6D is able to enhance joint transduction after intra-articular administration, evade AAV Nabs, without crossing the local barrier and entering the blood.

### Example 2

### AAV6 Induced a Highest Transduction Among AAV Serotypes in Joints

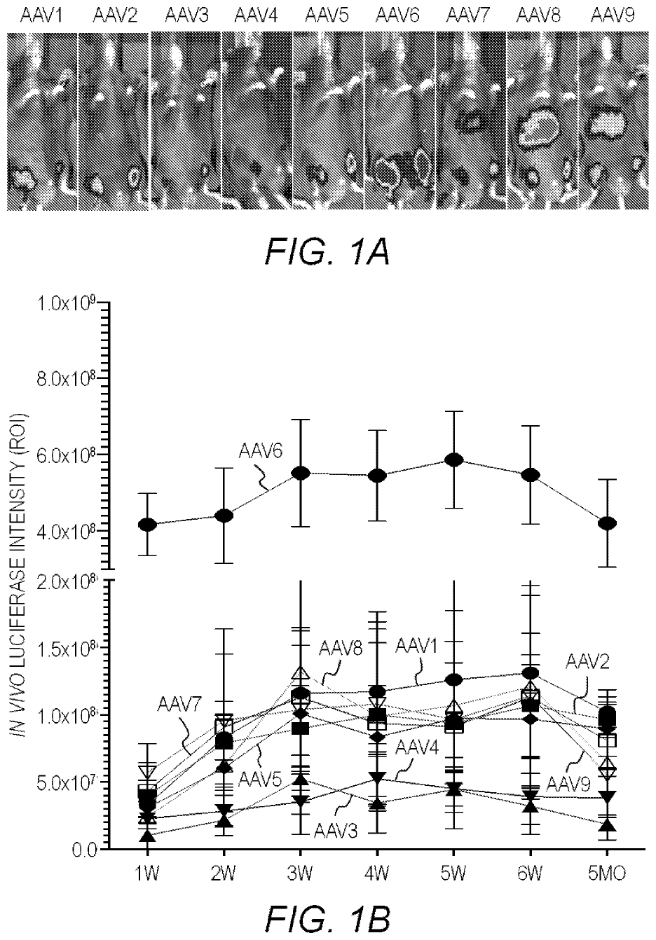

To verify the transduction efficiency in joints among AAV serotypes in mice, 5×10⁹ vg of AAV/luc vectors from serotypes 1 to 9 in 5 μl were injected in mouse knee joints. Imaging was performed at 1 w, 2 w, 3 w, 4 w, 5 w, 6 w, and 5 m after AAV injection (FIG. 1A). In the knee joint, generally for all serotypes of AAV vectors, the luciferase expression gradually increased the first 2 weeks, and remained stable after 3 weeks, at 5 months, the luciferase expression decreased to 20-30% of the peak level at week 3 post AAV injection (FIG. 1B). Among serotypes, AAV6 induced the highest transduction followed by AAV1, AAV2, AAV5, AAV7, AAV8 and AAV9, and AAV3 and 4 with the lowest transduction (p<0.05, FIG. 1B). Obvious luciferase expression was also detected in the liver in mice treated with IA injection of AAV7, AAV8, AAV9, but not in mice with other serotypes.

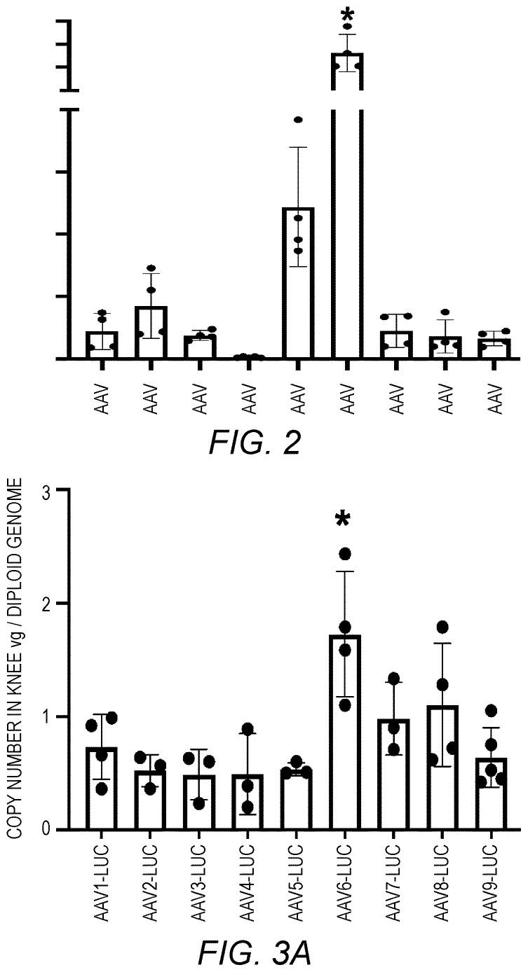

At month 6 post AAV IA injection, mice were sacrificed, the knee joints were collected and homogenized with 5× protein lysis buffer over night at 4° C., and an in vitro luciferase assay was performed. Consistent with in vivo imaging result, AAV6 had the highest luciferase activity followed by AAV5 and AAV2 in vitro (p<0.05, FIG. 2).

### Example 3

### AAV Genome Copy Number in Knee Joints and Livers

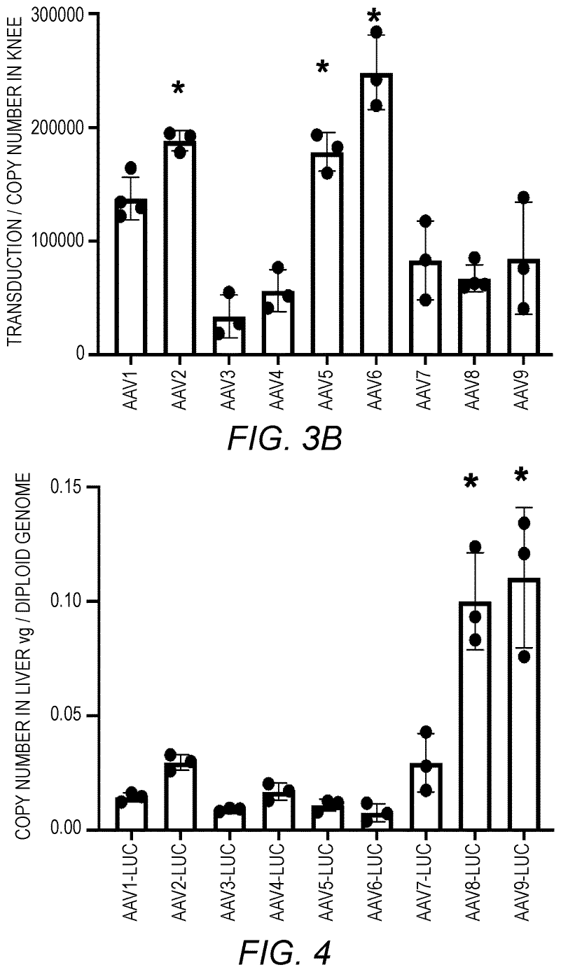

To study transduction efficiency in joint from different AAV serotypes after IA injection, we checked the viral genome copy number in AAV treated knee joints. The highest genome copy number was detected in AAV6 treated joints followed by AAV8, AAV7, AAV1 (FIG. 3A). After normalization of luciferase activity per AAV genome, AAV6 was still the best one to have high transgene expression followed by AAV2, AAV5 and AAV1 (FIG. 3B). These results suggest that AAV6 is the most efficient serotype to transduce the cells in joints. We also examined AAV copy number in the liver, consistent with the imaging data, higher genome copy number was detected in mice treated with AAV8 and AAV9, the lowest genome copy number in mice with AAV6 (FIG. 4).

### Example 4

### AAV is Able to Transduce Both Synoviocytes and Chondrocytes in the Joints

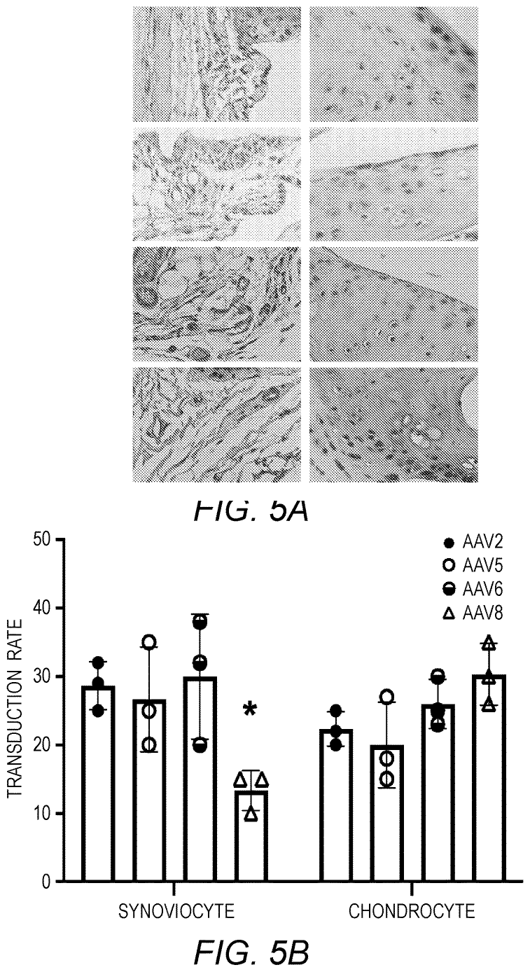

To determine which cell types can be transduced in the joint, at week 6 after IA injection of AAV/luc vectors from serotypes 2, 5, 6 and 8, mouse knee joints were fixed and stained by rabbit anti-luciferase primary antibody (FIG. 5A). All vectors were able to transduce 20-30% of chondrocytes without significant difference among serotypes (p>0.05). There was similar transduction efficiency in synoviocytes with serotypes AAV2, 5 and 6 at approximate 25%-30%. AAV8 induced much lower transduction in synoviocytes at 13.3±2.8% (p<0.05 when compared to serotypes 2, 5 and 6; FIG. 5B).

### Example 5

### VR1 Swapping Changes the AAV Transduction Profile in Joints

AAV VR1 has been demonstrated to play a role in transduction efficiency and neutralizing antibody (Nab) profile. Our study showed that AAV6 is the best serotype to transduce the joint as described above. In the next study, we swapped the VR1 from other serotypes into AAV6 capsid, or VR1 from AAV6 into other serotypes, and then investigated the transduction efficiency and immune profile. First, we examined the ratio of AAV capsid VP1, VP2 and VP3. After transfection of mutant plasmids into 293 cells, a western blot was performed for the cell lysate. As shown in FIG. 6, all three VPs were generated.

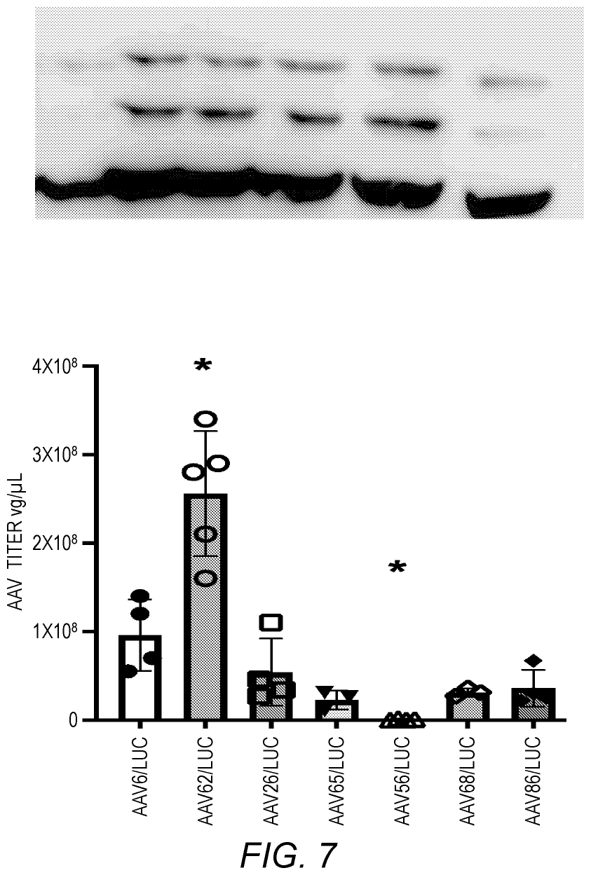

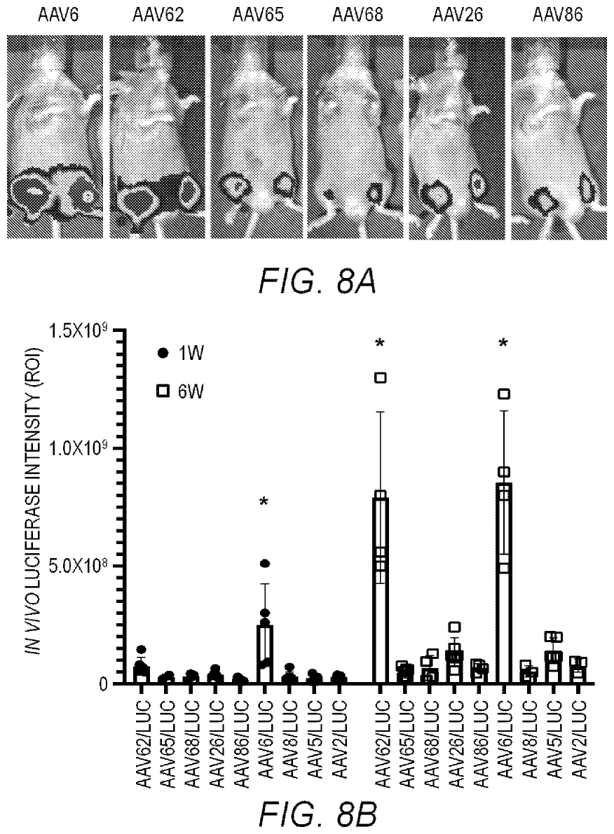

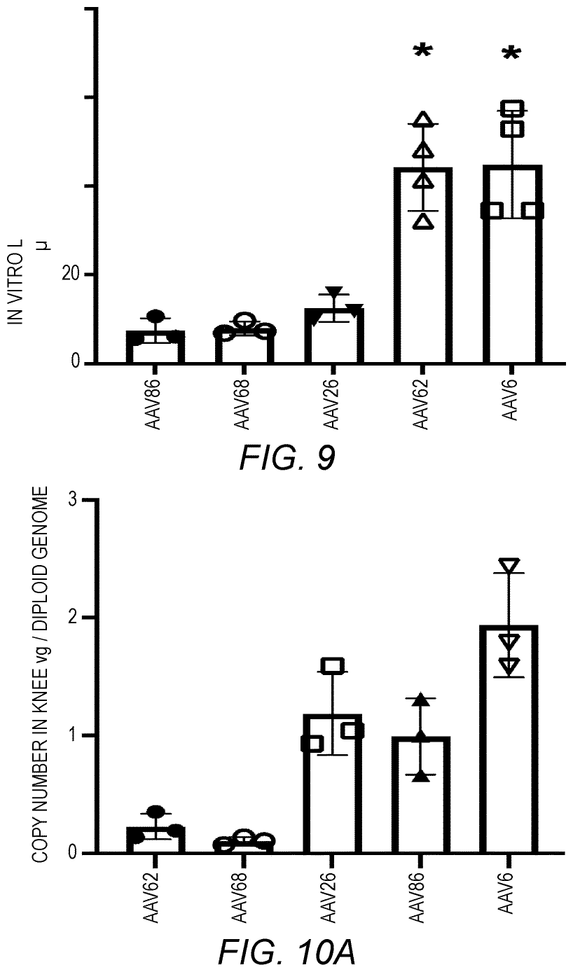

Next, we made AAV/luc vectors from these plasmids and quantified the virus titer in both supernatant and cell lysates using primers targeting the ITRs. The yield of AAV56 in which VR1 of AAV5 was replaced with AAV6 VR1 was the lowest and the highest yield of AAV vectors was AAV62. Other mutants produced a similar virus vector yield to AAV6 (FIG. 7). Then, we administered AAV/luc vectors at a dose of 5×10⁹ vg from these mutants and AAV6 into the knee joints of C57BL mice via IA injection. At week 1 and week 6 post AAV administration, imaging was carried out. At week 1, the highest transduction was observed with AAV6 (FIG. 8). At week 6, AAV 62 and 6 induced a similar transduction efficiency and higher than other mutants. However, AAV65 and AAV68 with the VR1 substitution from AAV5 or AAV8 had 7 and 9-fold lower transduction when compared to AAV6, respectively (p<0.05, FIG. 8). Generally, AAV26 and AAV86 with substitution of the VR1 from AAV6 had enhanced joint transduction than AAV2 or AAV8, respectively. We also collected the knee joints at week 6 post AAV injection from mice treated with AAV6 and mutants AAV62, AAV26, AAV68, and AAV86 vectors for luciferase analysis in vitro. Consistent with the result with imaging in mice, AAV 62 and AAV6 demonstrated 3-fold higher luciferase activity than AAV86, 68 and 26 (FIG. 9).

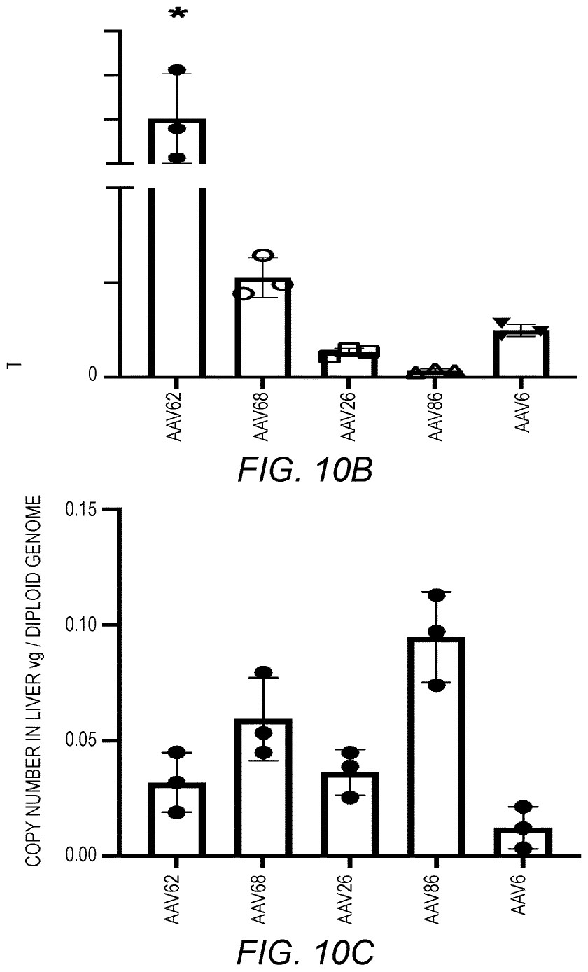

We also analyzed the genome copy number in the joints of mice treated with AAV vectors by qPCR using primers for luciferase. Five-fold higher copy number in mice receiving AAV6 was detected than that with AAV62 and AAV68, and 2-fold higher than that with AAV26 and AAV86 (FIG. 10A). After normalization to AAV genome copy number in the joints, the highest transgene expression was obtained in mice with AAV62 treatment followed by AAV86, AAV6, AAV26 and the lowest with AAV86 (FIG. 10B). Next, we examined the AAV genome copy number in the liver, the highest genome copy number was found in mice treated with AAV86 with ~0.1 AAV genome per cell, followed by AAV68, 26, AAV62, with AAV6 the lowest genome copy number in the liver (p<0.05, FIG. 10C). There was no difference for AAV genome copy number in the liver of mice treated with AAV62 and AAV6.

### Example 6

### Cell Tropisms of AAV Mutants in Joints

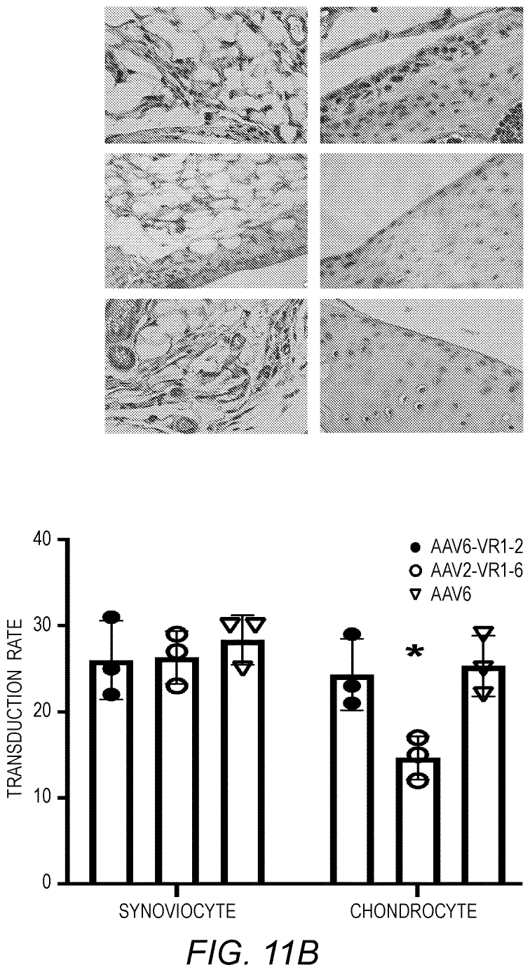

After collection of joints from mice treated with AAV6, AAV62 and AAV26 at week 6 post AAV injection, we sectioned the tissues and stained the slices with a rat anti-luciferase antibody. As shown in FIG. 11A and FIG. 11B, similar transduction in both synoviocytes and chondrocytes for AAV6 and AAV62 was observed, however, 1.5-fold lower transduction was seen in chondrocytes of mice with AAV26 when compared to AAV 6 or 62 (p<0.05) (FIG. 11B).

Neutralizing Antibody Pattern was Changed in Mutants with Substitution of VR1

It has been demonstrated that inflamed joint fluid contains high titer of Nabs which are able to block effective AAV transduction after IA administration. In this study, we studied the Nab profile in blood after AAV IA injection. Serum from mice treated with AAV62 had a Nab titer of 1:333 to AAV62, but 1:100 to AAV6. Similarly, the Nab titer in serum from AAV6 treated mice was 1:333 to AAV6 and 1:100 to AAV62. AAV62 or AAV6 did not generate Nabs against AAV26 and AAV2, while AAV26 and AAV2 still induced moderate neutralizing antibodies against AAV6 and AAV62 (1:10-100) (Table 6).

TABLE 6
The titer of neutralizing antibodies in blood
after AAV intra-articular injection.

Serum

| Vector | AAV62 | AAV26 | AAV6 | AAV2 |
|---|---|---|---|---|
| AAV26 | n | 333 | n | 333 |
| AAV62 | 333 | 33.3 | 100 | 33.3 |
| AAV6 | 100 | 100 | 333 | 10 |
| AAV2 | n | 100 | n | 333 |

### Mutation in AAV6 Capsid VR1 Enhanced Transduction

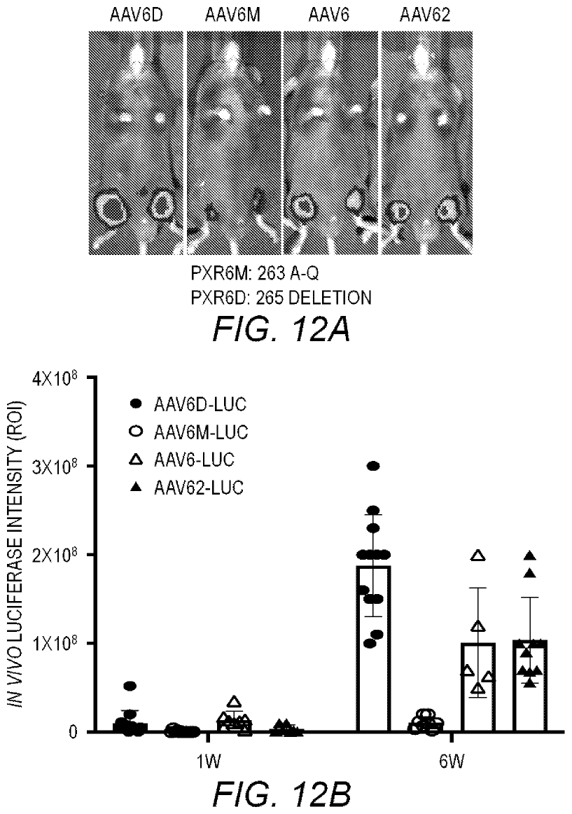

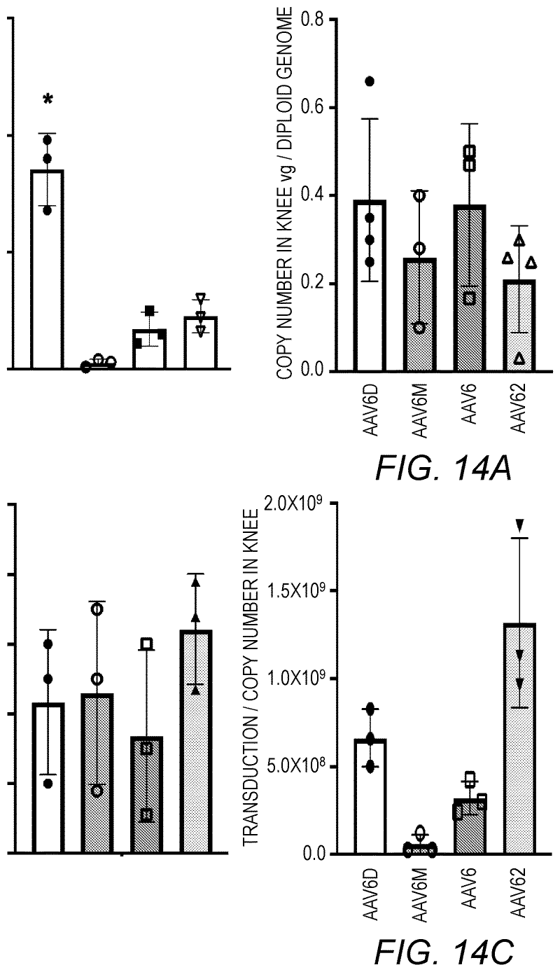

There are only two amino acids difference between VR1s from AAV2 and AAV6. AAV6 VR1 is one amino acid (threonine) insertion at the residue 265 of VP1 and another amino acid glutamine at residue 263 instead of alanine for AAV2 capsid. To study the role of two individual amino acids in AAV6 joint transduction, we designed two novel AAV capsids, the mutant AAV6D with a deletion of threonine at residue 265 and the mutant AAV6M with a mutation of glutamine to alanine at residue 263. After IA injection of AAV6 mutants in mice, imaging was taken at weeks 1 and 6. At week 1, the signal for all groups was relatively low, and there was no significant difference between different groups. At week 6, AAV6D mutant induced 2-fold higher transgene expression than AAV6 and AAV62, while AAV 6M mutant had a more than 5-fold lower transgene expression than AAV6 (FIG. 12). In agreement with the data in mouse imaging, 3.5-fold more luciferase activity was detected in cell lysate of knee joints treated with AAV6D when compared with AAV6 AAV (p<0.05, FIG. 13). However, the similar AAV genome copy number was found in the joints and the liver of mice regardless of AAV6 or AAV mutants (AAV6D and AAV6M) (FIG. 14A and FIG. 14B). When normalization with AAV gene copy number, both AAV6D and AAV62 induced much higher transgene expression than AAV6 in the joints (FIG. 14C).

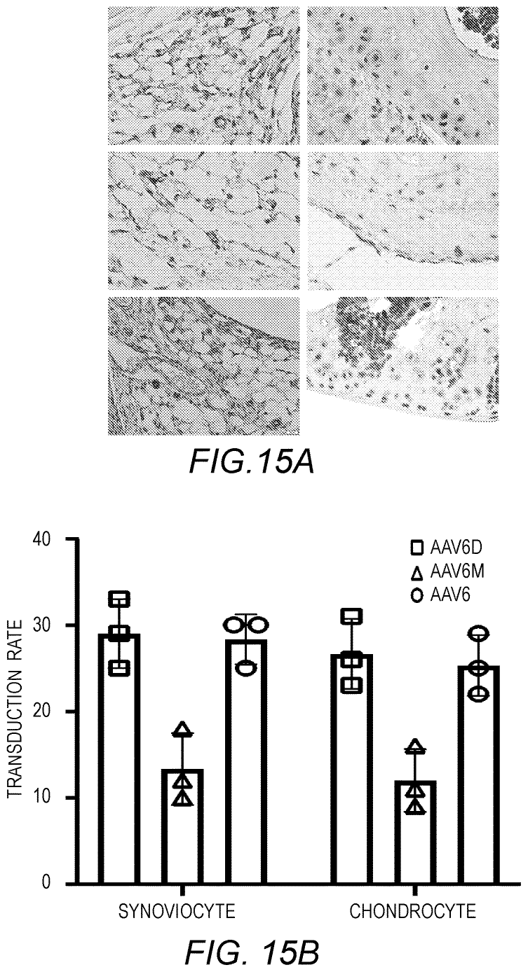

Additionally, AAV6D showed a similar cell tropism compared to AAV6 (p<0.05, FIG. 15A and FIG. 15B). 29.3±4.5% of synoviocytes and 26.8±4.1% of chondrocytes in the joints were transduced with AAV6D. Next, we analyzed the Nab profile in blood in mice treated with IA injection of AAV6 mutants. The administration of AAV6D generated 3-fold lower Nab titer than AAV6 (Table 7) and AAV6 Nab was less efficient to block AAV6D transduction (Table 7).

TABLE 7
The titer of neutralizing antibodies in blood
after AAV intra-articular injection.

Serum

| Vector | AAVD | AAVM | AAV6 | AAV62 |
|---|---|---|---|---|
| AAV6D | 33 | 10 | 33 | 10 |
| AAV6M | 33 | 33 | 33 | 100 |
| AAV6 | 10 | 10 | 100 | 33 |
| AAV62 | 10 | 10 | 33 | 100 |

The results from this study demonstrate that VR1 from AAV6 plays an important role in joint transduction efficiency and neutralizing antibody activity, and novel AAV mutants with rational engineering are able to enhance joint transduction after IA administration and evade AAV Nabs with decreased ability to cross the local barrier to enter the blood.

The foregoing examples are illustrative of the present invention and are not to be construed as limiting thereof. Although the invention has been described in detail with reference to preferred embodiments, variations and modifications exist within the scope and spirit of the invention as described and defined in the following claims.

### Sequences

SEQ ID NO: 1. AAV6
atggctgccgatggttatcttccagattggctcgaggacaacctctctgagggcattcgcgagtggtgggacttgaaacctggagccccgaa
acccaaagccaaccagcaaaagcaggacgacggccggggtctggtgcttcctggctacaagtacctcggacccttcaacggactcgaca
agggggagcccgtcaacgcggcggatgcagcggccctcgagcacgacaaggcctacgaccagcagctcaaagcgggtgacaatccgt
acctgcggtataaccacgccgacgccgagtttcaggagcgtctgcaagaagatacgtcttttgggggcaacctcgggcgagcagtcttcca
ggccaagaagagggttctcgaaccttttggtctggttgaggaaggtgctaagacggctcctggaaagaaacgtccggtagagcagtcgcc
acaagagccagactcctcctcgggcattggcaagacaggccagcagcccgctaaaaagagactcaattttggtcagactggcgactcaga
gtcagtccccgacccacaacctctcggagaacctccagcaacccccgctgctgtgggacctactacaatggcttcaggcggtggcgcacc
aatggcagacaataacgaaggcgccgacggagtgggtaatgcctcaggaaattggcattgcgattccacatggctgggcgacagagtcat
caccaccagcacccgaacatgggccttgcccacctataacaaccacctctacaagcaaatctccagtgcttcaacgggggccagcaacga
caaccactacttcggctacagcaccccctgggggtattttgatttcaacagattccactgccatttctcaccacgtgactggcagcgactcatc
aacaacaattggggattccggcccaagagactcaacttcaagctcttcaacatccaagtcaaggaggtcacgacgaatgatggcgtcacga
ccatcgctaataaccttaccagcacggttcaagtcttctcggactcggagtaccagttgccgtacgtcctcggctctgcgcaccagggctgc
ctccctccgttcccggcggacgtgttcatgattccgcagtacggctacctaacgctcaacaatggcagccaggcagtgggacggtcatcctt
ttactgcctggaatatttcccatcgcagatgctgagaacgggcaataactttaccttcagctacaccttcgaggacgtgcctttccacagcagc
tacgcgcacagccagagcctggaccggctgatgaatcctctcatcgaccagtacctgtattacctgaacagaactcagaatcagtccggaa
gtgcccaaaacaaggacttgctgtttagccgggggtctccagctggcatgtctgttcagcccaaaaactggctacctggaccctgttaccgg
cagcagcgcgtttctaaaacaaaaacagacaacaacaacagcaactttacctggactggtgcttcaaaatataaccttaatgggcgtgaatct
ataatcaaccctggcactgctatggcctcacacaaagacgacaaagacaagttctttcccatgagcggtgtcatgatttttggaaaggagagc
gccggagcttcaaacactgcattggacaatgtcatgatcacagacgaagaggaaatcaaagccactaaccccgtggccaccgaaagattt
gggactgtggcagtcaatctccagagcagcagcacagaccctgcgaccggagatgtgcatgttatgggagccttacctggaatggtgtgg
caagacagagacgtatacctgcagggtcctatttgggccaaaattcctcacacggatggacactttcacccgtctcctctcatgggcggcttt
ggacttaagcacccgcctcctcagatcctcatcaaaaacacgcctgttcctgcgaatcctccggcagagttttcggctacaaagtttgcttcat
tcatcacccagtattccacaggacaagtgagcgtggagattgaatgggagctgcagaaagaaaacagcaaacgctggaatcccgaagtgc
agtatacatctaactatgcaaaatctgccaacgttgatttcactgtggacaacaatggactttatactgagcctcgccccattggcacccgtta
cctcacccgtcccctgtaa
SEQ ID NO: 2. AAV6VP1
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPFGLVEEGAKTAPGKKRPVEQSPQEPDSSSGIGKTGQQPAKKRLN
FGQTGDSESVPDPQPLGEPPATPAAVGPTTMASGGGAPMADNNEGADGVGNASGNWH
CDSTWLGDRVITTSTRTWALPTYNNHLYKQISSASTGASNDNHYFGYSTPWGYFDFNRF
HCHFSPRDWQRLINNNWGFRPKRLNFKLFNIQVKEVTTNDGVTTIANNLTSTVQVFSDS
EYQLPYVLGSAHQGCLPPFPADVFMIPQYGYLTLNNGSQAVGRSSFYCLEYFPSQMLRT
GNNFTFSYTFEDVPFHSSYAHSQSLDRLMNPLIDQYLYYLNRTQNQSGSAQNKDLLFSR
GSPAGMSVQPKNWLPGPCYRQQRVSKTKTDNNNSNFTWTGASKYNLNGRESIINPGTA
MASHKDDKDKFFPMSGVMIFGKESAGASNTALDNVMITDEEEIKATNPVATERFGTVA
VNLQSSSTDPATGDVHVMGALPGMVWQDRDVYLQGPIWAKIPHTDGHFHPSPLMGGF
GLKHPPPQILIKNTPVPANPPAEFSATKFASFITQYSTGQVSVEIEWELQKENSKRWNPEV
QYTSNYAKSANVDFTVDNNGLYTEPRPIGTRYLTRPL
SEQ ID NO: 3. AAV6D
gaatgatggcgtcacgaccatcgctaataaccttaccagcacggttcaagtcttctcggactcggagtaccagttgccgtacgtcctcggctc
tgcgcaccagggctgcctccctccgttcccggcggacgtgttcatgattccgcagtacggctacctaacgctcaacaatggcagccaggca
gtgggacggtcatccttttactgcctggaatatttcccatcgcagatgctgagaacgggcaataactttaccttcagctacaccttcgaggacg
tgcctttccacagcagctacgcgcacagccagagcctggaccggctgatgaatcctctcatcgaccagtacctgtattacctgaacagaact
cagaatcagtccggaagtgcccaaaacaaggacttgctgtttagccgggggtctccagctggcatgtctgttcagcccaaaaactggctac
ctggaccctgttaccggcagcagcgcgtttctaaaacaaaaacagacaacaacaacagcaactttacctggactggtgcttcaaaatataac
cttaatgggcgtgaatctataatcaaccctggcactgctatggcctcacacaaagacgacaaagacaagttctttcccatgagcggtgtcatg
atttttggaaaggagagcgccggagcttcaaacactgcattggacaatgtcatgatcacagacgaagaggaaatcaaagccactaaccccg
tggccaccgaaagatttgggactgtggcagtcaatctccagagcagcagcacagaccctgcgaccggagatgtgcatgttatgggagcctt
acctggaatggtgtggcaagacagagacgtatacctgcagggtcctatttgggccaaaattcctcacacggatggacactttcacccgtctc
ctctcatgggcggctttggacttaagcacccgcctcctcagatcctcatcaaaaacacgcctgttcctgcgaatcctccggcagagttttcgg
ctacaaagtttgcttcattcatcacccagtattccacaggacaagtgagcgtggagattgaatgggagctgcagaaagaaaacagcaaacg
ctggaatcccgaagtgcagtatacatctaactatgcaaaatctgccaacgttgatttcactgtggacaacaatggactttatactgagcctcgc
cccattggcacccgttacctcacccgtcccctgtaa
SEQ ID NO: 4. AAV6DVP1
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPFGLVEEGAKTAPGKKRPVEQSPQEPDSSSGIGKTGQQPAKKRLN
FGQTGDSESVPDPQPLGEPPATPAAVGPTTMASGGGAPMADNNEGADGVGNASGNWH
CDSTWLGDRVITTSTRTWALPTYNNHLYKQISSASGASNDNHYFGYSTPWGYFDFNRF
HCHFSPRDWQRLINNNWGFRPKRLNFKLFNIQVKEVTTNDGVTTIANNLTSTVQVFSDS
EYQLPYVLGSAHQGCLPPFPADVFMIPQYGYLTLNNGSQAVGRSSFYCLEYFPSQMLRT
GNNFTFSYTFEDVPFHSSYAHSQSLDRLMNPLIDQYLYYLNRTQNQSGSAQNKDLLFSR
GSPAGMSVQPKNWLPGPCYRQQRVSKTKTDNNNSNFTWTGASKYNLNGRESIINPGTA
MASHKDDKDKFFPMSGVMIFGKESAGASNTALDNVMITDEEEIKATNPVATERFGTVA
VNLQSSSTDPATGDVHVMGALPGMVWQDRDVYLQGPIWAKIPHTDGHFHPSPLMGGF
GLKHPPPQILIKNTPVPANPPAEFSATKFASFITQYSTGQVSVEIEWELQKENSKRWNPEV
QYTSNYAKSANVDFTVDNNGLYTEPRPIGTRYLTRPL
SEQ ID NO: 5. AAV6M
Atggctgccgatggttatcttccagattggctcgaggacaacctctctgagggcattcgcgagtggtgggacttgaaacctggagccccga
aacccaaagccaaccagcaaaagcaggacgacggccggggtctggtgcttcctggctacaagtacctcggacccttcaacggactcgac
aagggggagcccgtcaacgcggcggatgcagcggccctcgagcacgacaaggcctacgaccagcagctcaaagcgggtgacaatcc
gtacctgcggtataaccacgccgacgccgagtttcaggagcgtctgcaagaagatacgtcttttgggggcaacctcgggcgagcagtcttc
caggccaagaagagggttctcgaaccttttggtctggttgaggaaggtgctaagacggctcctggaaagaaacgtccggtagagcagtcg
ccacaagagccagactcctcctcgggcattggcaagacaggccagcagcccgctaaaaagagactcaattttggtcagactggcgactca
gagtcagtccccgacccacaacctctcggagaacctccagcaacccccgctgctgtgggacctactacaatggcttcaggcggtggcgca
ccaatggcagacaataacgaaggcgccgacggagtgggtaatgcctcaggaaattggcattgcgattccacatggctgggcgacagagt
catcaccaccagcacccgaacatgggccttgcccacctataacaaccacctctacaagcaaatctccagtCAAtcaacgggggccagc
aacgacaaccactacttcggctacagcaccccctgggggtattttgatttcaacagattccactgccatttctcaccacgtgactggcagcga
ctcatcaacaacaattggggattccggcccaagagactcaacttcaagctcttcaacatccaagtcaaggaggtcacgacgaatgatggcgt
cacgaccatcgctaataaccttaccagcacggttcaagtcttctcggactcggagtaccagttgccgtacgtcctcggctctgcgcaccagg
gctgcctccctccgttcccggcggacgtgttcatgattccgcagtacggctacctaacgctcaacaatggcagccaggcagtgggacggtc
atccttttactgcctggaatatttcccatcgcagatgctgagaacgggcaataactttaccttcagctacaccttcgaggacgtgcctttccac
agcagctacgcgcacagccagagcctggaccggctgatgaatcctctcatcgaccagtacctgtattacctgaacagaactcagaatcagtc
cggaagtgcccaaaacaaggacttgctgtttagccgggggtctccagctggcatgtctgttcagcccaaaaactggctacctggaccctgtt
accggcagcagcgcgtttctaaaacaaaaacagacaacaacaacagcaactttacctggactggtgcttcaaaatataaccttaatgggcgt
gaatctataatcaaccctggcactgctatggcctcacacaaagacgacaaagacaagttctttcccatgagcggtgtcatgatttttggaaag
gagagcgccggagcttcaaacactgcattggacaatgtcatgatcacagacgaagaggaaatcaaagccactaaccccgtggccaccga
aagatttgggactgtggcagtcaatctccagagcagcagcacagaccctgcgaccggagatgtgcatgttatgggagccttacctggaatg
gtgtggcaagacagagacgtatacctgcagggtcctatttgggccaaaattcctcacacggatggacactttcacccgtctcctctcatgggc
ggctttggacttaagcacccgcctcctcagatcctcatcaaaaacacgcctgttcctgcgaatcctccggcagagttttcggctacaaagtttg
cttcattcatcacccagtattccacaggacaagtgagcgtggagattgaatgggagctgcagaaagaaaacagcaaacgctggaatcccg
aagtgcagtatacatctaactatgcaaaatctgccaacgttgatttcactgtggacaacaatggactttatactgagcctcgccccattggcac
ccgttacctcacccgtcccctgtaa
SEQ ID NO: 6. AAV6MVP1
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPFGLVEEGAKTAPGKKRPVEQSPQEPDSSSGIGKTGQQPAKKRLN
FGQTGDSESVPDPQPLGEPPATPAAVGPTTMASGGGAPMADNNEGADGVGNASGNWH
CDSTWLGDRVITTSTRTWALPTYNNHLYKQISSQSTGASNDNHYFGYSTPWGYFDFNRF
HCHFSPRDWQRLINNNWGFRPKRLNFKLFNIQVKEVTTNDGVTTIANNLTSTVQVFSDS
EYQLPYVLGSAHQGCLPPFPADVFMIPQYGYLTLNNGSQAVGRSSFYCLEYFPSQMLRT
GNNFTFSYTFEDVPFHSSYAHSQSLDRLMNPLIDQYLYYLNRTQNQSGSAQNKDLLFSR
GSPAGMSVQPKNWLPGPCYRQQRVSKTKTDNNNSNFTWTGASKYNLNGRESIINPGTA
MASHKDDKDKFFPMSGVMIFGKESAGASNTALDNVMITDEEEIKATNPVATERFGTVA
VNLQSSSTDPATGDVHVMGALPGMVWQDRDVYLQGPIWAKIPHTDGHFHPSPLMGGF
GLKHPPPQILIKNTPVPANPPAEFSATKFASFITQYSTGQVSVEIEWELQKENSKRWNPEV
QYTSNYAKSANVDFTVDNNGLYTEPRPIGTRYLTRPL
SEQ ID NO: 7. AAV62
atggctgccgatggttatcttccagattggctcgaggacaacctctctgagggcattcgcgagtggtgggacttgaaacctggagccccgaa
acccaaagccaaccagcaaaagcaggacgacggccggggtctggtgcttcctggctacaagtacctcggacccttcaacggactcgaca
agggggagcccgtcaacgcggcggatgcagcggccctcgagcacgacaaggcctacgaccagcagctcaaagcgggtgacaatccgt
acctgcggtataaccacgccgacgccgagtttcaggagcgtctgcaagaagatacgtcttttgggggcaacctcgggcgagcagtcttcca
ggccaagaagagggttctcgaaccttttggtctggttgaggaaggtgctaagacggctcctggaaagaaacgtccggtagagcagtcgcc
acaagagccagactcctcctcgggcattggcaagacaggccagcagcccgctaaaaagagactcaattttggtcagactggcgactcaga
gtcagtccccgacccacaacctctcggagaacctccagcaacccccgctgctgtgggacctactacaatggcttcaggcggtggcgcacc
aatggcagacaataacgaaggcgccgacggagtgggtaatgcctcaggaaattggcattgcgattccacatggctgggcgacagagtcat
caccaccagcacccgaacatgggccttgcccacctataacaaccacctctacaagcaaatctccAGCCAATCAGGAGCCT
CGaacgacaaccactacttcggctacagcaccccctgggggtattttgatttcaacagattccactgccatttctcaccacgtgactggcag
cgactcatcaacaacaattggggattccggcccaagagactcaacttcaagctcttcaacatccaagtcaaggaggtcacgacgaatgatg
gcgtcacgaccatcgctaataaccttaccagcacggttcaagtcttctcggactcggagtaccagttgccgtacgtcctcggctctgcgcacc
agggctgcctccctccgttcccggcggacgtgttcatgattccgcagtacggctacctaacgctcaacaatggcagccaggcagtgggac
ggtcatccttttactgcctggaatatttcccatcgcagatgctgagaacgggcaataactttaccttcagctacaccttcgaggacgtgccttt
ccacagcagctacgcgcacagccagagcctggaccggctgatgaatcctctcatcgaccagtacctgtattacctgaacagaactcagaatc
agtccggaagtgcccaaaacaaggacttgctgtttagccgggggtctccagctggcatgtctgttcagcccaaaaactggctacctggacc
ctgttaccggcagcagcgcgtttctaaaacaaaaacagacaacaacaacagcaactttacctggactggtgcttcaaaatataaccttaatgg
gcgtgaatctataatcaaccctggcactgctatggcctcacacaaagacgacaaagacaagttctttcccatgagcggtgtcatgatttttgga
aaggagagcgccggagcttcaaacactgcattggacaatgtcatgatcacagacgaagaggaaatcaaagccactaaccccgtggccac
cgaaagatttgggactgtggcagtcaatctccagagcagcagcacagaccctgcgaccggagatgtgcatgttatgggagccttacctgga
atggtgtggcaagacagagacgtatacctgcagggtcctatttgggccaaaattcctcacacggatggacactttcacccgtctcctctcatg
ggcggctttggacttaagcacccgcctcctcagatcctcatcaaaaacacgcctgttcctgcgaatcctccggcagagttttcggctacaaag
tttgcttcattcatcacccagtattccacaggacaagtgagcgtggagattgaatgggagctgcagaaagaaaacagcaaacgctggaatcc
cgaagtgcagtatacatctaactatgcaaaatctgccaacgttgatttcactgtggacaacaatggactttatactgagcctcgccccattggc
acccgttacctcacccgtcccctgtaa
SEQ ID NO: 8. AAV62VP1
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPFGLVEEGAKTAPGKKRPVEQSPQEPDSSSGIGKTGQQPAKKRLN
FGQTGDSESVPDPQPLGEPPATPAAVGPTTMASGGGAPMADNNEGADGVGNASGNWH
CDSTWLGDRVITTSTRTWALPTYNNHLYKQISSQSGASNDNHYFGYSTPWGYFDFNRF
HCHFSPRDWQRLINNNWGFRPKRLNFKLFNIQVKEVTTNDGVTTIANNLTSTVQVFSDS
EYQLPYVLGSAHQGCLPPFPADVFMIPQYGYLTLNNGSQAVGRSSFYCLEYFPSQMLRT
GNNFTFSYTFEDVPFHSSYAHSQSLDRLMNPLIDQYLYYLNRTQNQSGSAQNKDLLFSR
GSPAGMSVQPKNWLPGPCYRQQRVSKTKTDNNNSNFTWTGASKYNLNGRESIINPGTA
MASHKDDKDKFFPMSGVMIFGKESAGASNTALDNVMITDEEEIKATNPVATERFGTVA
VNLQSSSTDPATGDVHVMGALPGMVWQDRDVYLQGPIWAKIPHTDGHFHPSPLMGGF
GLKHPPPQILIKNTPVPANPPAEFSATKFASFITQYSTGQVSVEIEWELQKENSKRWNPEV
QYTSNYAKSANVDFTVDNNGLYTEPRPIGTRYLTRPL
SEQ ID NO: 9. AAV2VR1
aagcaaatctccAGCCAATCAGGAGCCTCGaacgacaac
SEQ ID NO: 10. AAV2VR1
KQISSQSGASNDN
SEQ ID NO: 11. AAV5VR1
cgagagatcaaaagcggctccgtcgacggaagcaacgccaac
SEQ ID NO: 12. AAV5VR1
REIKSGSVDGSNAN
SEQ ID NO: 13. AAV6VR1
aagcaaatctccAgtgcttcaacgggggccagcaacgacaac
SEQ ID NO: 14. AAV6VR1
KQISSASTGASNDN
SEQ ID NO: 15. AAV8VR1
aagcaaatctccaacgggacatcgggaggagccaccaacgacaac
SEQ ID NO: 16. AAV8VR1
KQISNGTSGGATNDN

1. An adenovirus-associated virus (AAV) capsid protein comprising a capsid protein amino acid sequence from AAV6, wherein the variable region 1 (VR1) loop comprising amino acid residues 258 to 271 of AAV6 capsid protein is modified by deletion and/or substitution of one or more amino acid residues, wherein the capsid protein comprising the modification provides to an AAV vector comprising the capsid protein increased transduction efficiency and increased ability to evade neutralizing antibodies when administered intra-articularly, relative to an AAV vector comprising an unmodified AAV6 capsid protein.

2. The capsid protein of claim 1, wherein one or more of amino acid residues 258 to 271 of AAV6 capsid protein are deleted, optionally wherein amino acid residue threonine 265 of AAV6 capsid protein is deleted.

3. (canceled)

4. The capsid protein of claim 1, wherein one or more of amino acid residues 258 to 271 of AAV6 capsid protein are substituted, optionally wherein the VR1 loop of AAV6 capsid protein is substituted with the VR1 loop of AAV2 capsid protein (amino acid residues 258-270), optionally wherein amino acid residue alanine 263 of AAV6 capsid protein is substituted with glutamine.

5-7. (canceled)

8. The capsid protein of claim 1, wherein transduction of joints is increased at least about 10% relative to an AAV vector comprising a capsid protein that does not contain the modification.

9. The capsid protein of claim 1, wherein the capsid protein comprises the amino acid sequence of any one of SEQ ID NO:4, SEQ ID NO:6, or SEQ ID NO:8, or a sequence at least 90% identical thereto.

10-11. (canceled)

12. A polynucleotide encoding the capsid protein of claim 1.

13. The polynucleotide of claim 12, wherein the polynucleotide comprises the nucleotide sequence of one of SEQ ID NOS: 3, 5, or 7 or a sequence at least 90% identical thereto.

14. An AAV capsid comprising the capsid protein of claim 1.

15. An AAV vector comprising:
(a) the AAV capsid of claim 14; and
(b) a nucleic acid comprising a recombinant viral template,
wherein the nucleic acid is encapsidated by the AAV capsid.

16. The AAV vector of claim 15, wherein the template comprises a heterologous nucleic acid of interest.

17. The AAV vector of claim 15, wherein the template comprises a promoter, optionally wherein the promoter is tissue specific, optionally wherein the promoter is specific to chondrocytes and/or synoviocytes.

18-19. (canceled)

20. The AAV vector of claim 15, wherein the AAV vector exhibits minimized leakage from a joint into the blood and/or liver.

21. A pharmaceutical composition comprising the AAV vector of claim 15 in a pharmaceutically acceptable carrier.

22. A method of delivering a nucleic acid to a cell of a joint, the method comprising contacting the cell with the AAV vector of claim 15 under conditions sufficient for the nucleic acid to enter the cell, optionally wherein the joint is a knee, knuckle, or shoulder joint.

23. A method of delivering a nucleic acid to a joint of a subject, the method comprising administering intra-articularly to the subject the AAV vector of claim 15.

24. The method of claim 23, wherein the subject is a human subject, optionally wherein the subject has or is at risk of developing a joint issue, optionally wherein the joint issue is selected from a group consisting of joint disease, joint pain, joint inflammation, and joint injury.

25-26. (canceled)

27. The method of claim 23, wherein the subject has or is at risk of developing arthritis, optionally wherein the arthritis is selected from a group consisting of osteoarthritis, juvenile arthritis, reactive arthritis, gout, psoriatic arthritis, fibromyalgia, ankylosing spondylitis or rheumatoid arthritis, Felty's syndrome, diffuse idiopathic skeletal hyperostosis (DISH), Behçet's disease, inflammatory arthritis, mixed connective tissue disease, infectious arthritis, Granulomatosis with polyangiitis (GPA), Mixed Connective Tissue disease (MCTD), myositis (dermatomyositis, polymyositis), Paget's disease, Pseudogout, polymyalgia rheumatica with giant cell arteritis, Raynaud's Phenomenon, scleroderma, systemic lupus erythematosus, and Sjögren syndrome.

28. (canceled)

29. A method for treating and/or delaying onset of arthritis in a subject in need thereof, comprising administering to the subject an effective amount of the AAV vector of claim 15, thereby treating and/or delaying onset of arthritis in the subject, optionally wherein the AAV vector or pharmaceutical composition is administered via intra-articular injection.

30. The method of claim 29, wherein the arthritis is selected from a group consisting of osteoarthritis, juvenile arthritis, reactive arthritis, gout, psoriatic arthritis, fibromyalgia, ankylosing spondylitis or rheumatoid arthritis, Felty's syndrome, diffuse idiopathic skeletal hyperostosis (DISH), Behçet's disease, inflammatory arthritis, mixed connective tissue disease, infectious arthritis, Granulomatosis with polyangiitis (GPA), Mixed Connective Tissue disease (MCTD), myositis (dermatomyositis, polymyositis), Paget's disease, Pseudogout, polymyalgia rheumatica with giant cell arteritis, Raynaud's Phenomenon, scleroderma, systemic lupus erythematosus, and Sjögren syndrome.

31-35. (canceled)

36. A method of producing a recombinant AAV particle, comprising providing to a cell permissive for AAV replication:
(a) a recombinant AAV template comprising (i) a heterologous nucleic acid, and (ii) at least one inverted terminal repeat; and
(b) a polynucleotide comprising replication protein coding sequence(s) and sequence(s) encoding the capsid protein of claim 1;
under conditions sufficient for the replication and packaging of the recombinant AAV template;
whereby recombinant AAV particles are produced in the cell.
